(19) United States
(12) Patent Application Publication
(10) Pub. No.: US 2023/0372608 A1
(43) Pub. Date: Nov. 23, 2023

(54) NON-ADHESIVE IV START KIT

(71) Applicant: Mary Pickett, Salisbury, NC (US)

(72) Inventor: Mary Pickett, Salisbury, NC (US)

(21) Appl. No.: 17/746,254

(22) Filed: May 17, 2022

(51) Int. Cl. A61M 5/158

(52) U.S. Cl. CPC A61M 5/158; A61M 2025/0266

(57) ABSTRACT

The invention is directed to a method of securing a catheter to the body of patient without the use of adhesive materials. The method includes applying an anchor (e.g., non-adhesive tape) to the patient's skin adjacent to the site where the catheter needle will be inserted by a medical practitioner. The catheter needle is then inserted into the patient's vein. In some embodiments, an antimicrobial patch can be applied over the needle at the injection site. A non-adhesive covering is then applied over the system (e.g., the inserted needle, non-adhesive tape, and antimicrobial patch). In this way, the system is maintained in position, the catheter is accessible as needed, and pain is reduced when the system is disassembled.

### TECHNICAL FIELD

The presently disclosed subject matter is generally directed to a system and method of inserting and securing an IV line in a patient.

### BACKGROUND

One of the most frequently performed medical procedures is the insertion of a needle into a live human body for the purpose of drawing blood from a vessel, delivering fluids and drugs, inserting a catheter, performing diagnostic tests, and the like. Conventionally, after a needle is inserted through a patient's skin into a vein, it must be secured to prevent movement and slippage. Commonly, adhesive surgical tape is used to hold the catheter hub or tubing connected to the needle to the patient's skin. However, the adhesive tape is ineffective to anchor the needle and prevent catheter movement permanently and securely. Specifically, the adhesive tape frequently allows the needle to move within a vein, causing irritation and requiring removal of the catheter and reinsertion at a different location. In addition, prolonged contact with the adhesive tape can cause skin irritation. After the infusion or other procedure is concluded, the adhesive tape is removed from the patient's skin. Despite efforts to minimize trauma or discomfort to the patient, removal of the adhesive tape often injures the patient's skin and causes the patient localized pain. For example, removal of the adhesive tape can tear or otherwise cause injury to the skin, which necessitates additional treatment. It would therefore be beneficial to provide a system and method of inserting and securing an IV line that overcomes the shortcomings of the prior art.

### SUMMARY

In some embodiments, the presently disclosed subject matter is directed to a method of securely inserting a catheter in a vessel of a patient. Specifically, the method comprising applying an anchor on a patient's skin, adjacent to a vessel, wherein the anchor is defined as a portion of non-adhesive material. The method further includes inserting a catheter needle into the vessel at an injection site such that the needle partially overlays the anchor. The method includes wrapping an outer covering around the anchor and catheter needle at the injection site, wherein the outer covering is defined as a portion of non-adhesive material, such that the catheter is securely inserted in the patient's vessel.

In some embodiments, the method includes applying an antimicrobial patch over the injection site after the needle has been inserted and prior to wrapping the outer covering.

In some embodiments, the anchor and outer covering each have a peel strength of less than 0.01 N/20 mm.

In some embodiments, the anchor and outer covering do not adhere to the skin of the patient.

In some embodiments, the anchor comprises a bacteriostatic or bactericidal agent.

In some embodiments, the anchor is constructed from hydrocolloid, acrylic PSA, silicone, elastomer, rubber, polyurethane, or combinations thereof.

In some embodiments, the anchor has a length and width of about 0.5-4 inches and a thickness of at least about 0.001 inches.

In some embodiments, the catheter includes a needle, a hub, and tubing.

In some embodiments, the antimicrobial patch includes chlorhexidine base, chlorhexidine acetate, chlorhexidine gluconate, EDTA, silver sulfadiazine, taurolidine, or combinations thereof.

In some embodiments, the outer covering is transparent.

In some embodiments, the outer covering is semi-transparent.

In some embodiments, the outer covering comprises saran, PVC, polyurethane, nylon polyurethane, nylon, polyethylene terephthalate, or combinations thereof.

In some embodiments, the outer covering is flexible.

In some embodiments, the outer covering wraps fully around a patient's limb.

In some embodiments, the outer cover releasably attaches to itself.

In some embodiments,

In some embodiments, the presently disclosed subject matter is directed to a kit for performing the disclosed method. Specifically, the kit comprises a plurality of anchors that vary in size, shape, composition, or combinations thereof. The kit also includes a plurality of catheters that vary in size, shape, purpose, composition, or combinations thereof. The kit further includers a plurality of outer coverings that vary in size, shape, composition, or combinations thereof.

In some embodiments, the kit further includes a plurality of antimicrobial patches that vary in size, shape, composition, or combinations thereof.

In some embodiments, the outer covering is transparent or semi-transparent.

In some embodiments, the outer covering comprises saran, PVC, polyurethane, nylon polyurethane, nylon, polyethylene terephthalate, or combinations thereof.

In some embodiments, the anchor comprises a bacteriostatic or bactericidal agent.

### BRIEF DESCRIPTION OF THE DRAWINGS

FIG. 1 is a schematic illustrating steps in accordance with one method of securing a catheter to a patient vein in accordance with some embodiments of the presently disclosed subject matter.

FIG. 2a is a top plan view of a patient arm with an applied anchor in accordance with some embodiments of the presently disclosed subject matter.

FIG. 2b is a perspective view of an anchor in accordance with some embodiments of the presently disclosed subject matter.

FIG. 2c is a side plan view of an anchor in accordance with some embodiments of the presently disclosed subject matter.

FIGS. 3a-3f are top plan views of anchors in varying shapes in accordance with some embodiments of the presently disclosed subject matter.

FIG. 4a is a top plan view of a patient arm with an anchor and catheter in accordance with some embodiments of the presently disclosed subject matter.

FIG. 4b is a side view of a catheter and anchor in use in accordance with some embodiments of the presently disclosed subject matter.

FIG. 5a is a top plan view of a patient arm with an anchor, catheter, and antimicrobial patch in accordance with some embodiments of the presently disclosed subject matter.

FIG. 5b is a side view of an antimicrobial patch in place on an injection site in accordance with some embodiments of the presently disclosed subject matter.

FIG. 5c is a perspective view of an antimicrobial patch in accordance with some embodiments of the presently disclosed subject matter.

FIG. 5d is a top plan view of an antimicrobial patch in accordance with some embodiments of the presently disclosed subject matter.

FIG. 6a is a top plan view of a patient arm with an anchor, catheter, antimicrobial patch and outer covering in accordance with some embodiments of the presently disclosed subject matter.

FIG. 6b is a top plan view of a patient arm with an anchor, catheter, and outer covering in accordance with some embodiments of the presently disclosed subject matter.

FIG. 7 is a schematic of a kit comprising a plurality of anchors, catheters, outer coverings, and optional antimicrobial patches in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments+/−20%, in some embodiments+/−10%, in some embodiments+/−5%, in some embodiments+/−1%, in some embodiments+/−0.5%, and in some embodiments +/−0.1%, from the specified amount, as such variations are appropriate in the disclosed packages and methods.

As used herein, the term “and/or” includes any and all combinations of one or more of the associated listed items.

Relative terms such as “below” or “above” or “upper” or “lower” or “horizontal” or “vertical” may be used herein to describe a relationship of one element, layer, or region to another element, layer, or region as illustrated in the drawing figures. It will be understood that these terms and those discussed above are intended to encompass different orientations of the device in addition to the orientation depicted in the drawing figures.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The presently disclosed subject matter is generally directed to a method of securing a catheter to the body of patient without the use of adhesive materials (e.g., adhesive tape). The term “catheter” as used herein refers to any medical tubing that includes a needle at one end for disposition in or along the body of a patient. As illustrated in the schematic of FIG. 1, method 5 includes applying an anchor (e.g., non-adhesive tape) to the patient's skin adjacent to the site where the catheter needle will be inserted by the medical practitioner at step 10. The catheter needle is then inserted into the patient's vein at step 15. In some embodiments, an antimicrobial patch can be applied over the needle at the injection site at step 20. A non-adhesive covering is then applied over the system (e.g., the inserted needle, non-adhesive tape, and antimicrobial patch) at the injection site at step 25. In this way, the system is maintained in position, the catheter is accessible as needed, and pain is reduced when the system is disassembled. Importantly, the disclosed system and method can be 100 percent free from adhesive materials.

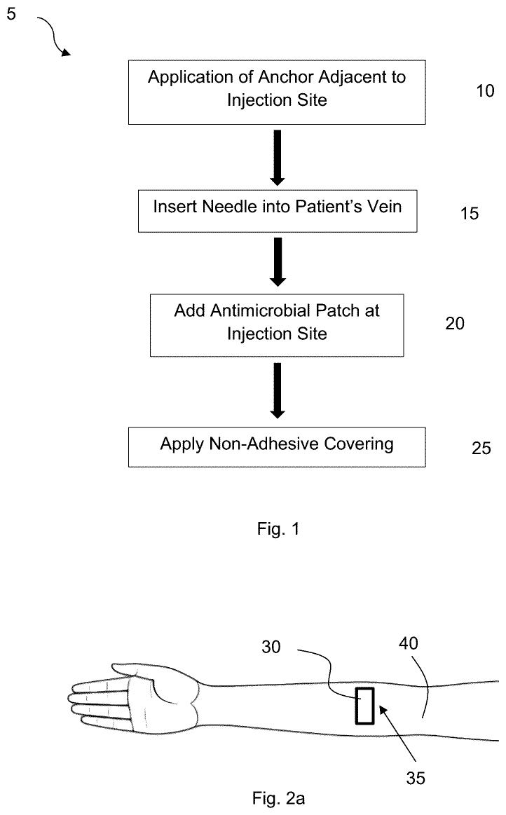

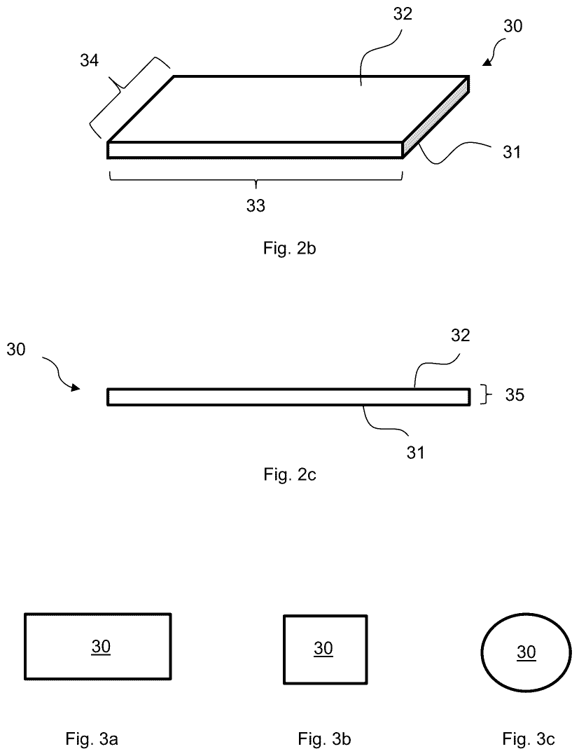

As set forth above, the disclosed method includes a first step of applying an anchor adjacent to the injection site in a patient. As shown in FIG. 2a, anchor 30 is positioned adjacent to injection site 35 in patient 40. The term “injection site” refers to the location on the patient's body where a needle is inserted to access a vein. Stated another way, the injection site is the point at which a needle or other puncturing device makes contact with the patient. Although the arm of patient 40 is shown in the figures, the injection site can be any location on the patient's body. The term “anchor” refers to an element that acts to support the catheter needle during use. One embodiment of anchor 30 comprising first side 31 and opposed side 32 is illustrated in FIGS. 2b and 2c. The first side comes into direct contact with the patient's skin during use, while second side 32 faces the exterior environment. The anchor therefore provides support for the needle once inserted (e.g., the portion that remains outside the body) and acts as a landmark for the vessel to be accessed.

Importantly, anchor 30 is formed from non-adhesive materials. The term “non-adhesive” refers to a material that has a low affinity (10% or less) or no affinity for adhering to a patient's skin. For example, in some embodiments, a non-adhesive material does not adhere to the skin. In some embodiments, a non-adhesive material has a peel strength of less than 0.1 N/20 mm, less than 0.01 N/20 mm, or less than 0.001 N/20 mm. The “peel strength” refers to the strength required to remove the material from the skin. In some embodiments, both the first and second sides 31, 32 are non-adhesive (e.g., the anchor is about 100% non-adhesive). In other embodiments, only the first side is non-adhesive, which allows the needle to be more securely retained via contact with the adhesive or partially-adhesive second side. In some embodiments, the first and/or second sides of the anchor are 100 percent non-adhesive. In other embodiments, the first and/or second side of the anchor are about 90-99.9 percent non-adhesive.

Anchor 30 can be constructed from any suitable material, such as (but not limited to) hydrocolloid, plastic, paper, paperboard, acrylic PSA, silicone, elastomer, rubber, polyurethane, foam, fabric, textile, felt, plastic, or combinations thereof. The anchor can therefore take the form of a non-adhesive pad, tape, cushion, swab, etc. In some embodiments, the anchor (or at least the first side 31 of the anchor) can include padding or soft material to provide an added layer of protection to the patient's skin.

In some embodiments, the anchor includes a bacteriostatic or bactericidal agent to destroy any harmful organisms present in deep pores, hair follicles, or sweat glands of the skin. The term “bacteriostatic” refers to an agent that stops bacteria from reproducing. The term “bactericidal” refers to an agent that kills bacteria. Any suitable bacteriostatic or bactericidal material can be used, such as (but not limited to) Betadine®, Polysporin®, chloramphenicol, clindamycin, ethambutol, lincosamides, macrolide, nitrofurantoin, novobiocin, oxazolidinone, spectinomycin, sulfonamide, iodine, alcohol, peroxide, iodine, silver, silver nitrate, mercury chloride, copper sulfate, heavy metals, heavy metal salts, and the like. As a result, bacteria is prevented from reaching the injection site through or under the anchor.

The anchor can be flexile in nature. The term “flexible” refers to the ability to bend easily, without damage or tearing. In this way, the anchor can adjust and adapt when the patient moves their body.

The anchor can have any suitable dimensions, such as length 33 and/or width 34 of about 0.5-4 inches (e.g., at least/no more than about 0.5, 1, 1.5, 2, 2.5, 3, 3.5, or 4 inches). The anchor can further include thickness 35 of at least about (or no more than about) 0.001, 0.01, 0.1, 0.2, 0.3, 0.4, or 0.5 inches. It should be appreciated that the length, width, and thickness of the anchor are not limited, and can be configured outside the ranges given herein.

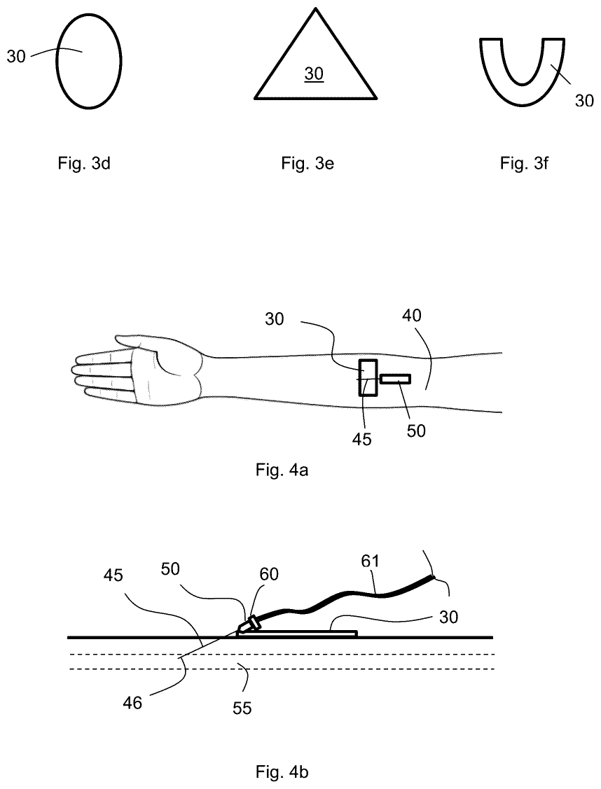

Anchor 30 can be constructed in any suitable shape, such as rectangular, square, circular, oval, triangular, U-shaped, and the like, as shown in FIGS. 3a-3f. Any shape can be used.

The anchor acts as a marker of the injection site for the medical practitioner. In addition, the anchor cushions and supports the needle of the catheter once inserted into the patient's vein. In some embodiments, the side of the anchor that contacts the needle can be tacky, partially adhesive, or adhesive to retain the needle in position and keep it from moving. In other embodiments, however, the second side of the anchor is non-adhesive.

After the anchor is positioned on the skin of the patient, needle 45 of catheter 50 is then inserted into a suitable vessel 55 in the patient. Typically, the vessel will be positioned at or near the location of adhesive tape 30, as shown in FIGS. 4a and 4b. Specifically, sharp tip 46 of needle 45 pierces the skin of patient 40 until it reaches vessel 55. The needle traverses a distance through the subcutaneous connective tissue from the skin puncture site until it reaches the vessel (e.g., a vein). The distance can be about 0.01-0.7 inches (e.g., at least/no more than about 0.01, 0.1, 0.2, 0.3, 0.4, 0.5, 0.6, or 0.7 inches). However, the distance the needle traverses can be any suitable distance.

The needle can then access the vessel, such as to withdraw blood, add fluids, and the like. In some embodiments, the catheter can include hub 60 and tubing 61 that connects to a source of infusion liquid (not shown). In some embodiments, the hub can be configured as a valve, a catheter body, a Luer-type connector, a threaded fitting, a friction fitting, and the like. Thus, the distal end of a catheter can be inserted into a patient at an injection site. The proximal end of the catheter can then connect to an IV bag or other element via hub 60. The catheter, needle, tubing, and hub can vary in shape, size, and configuration from one manufacturer to another.

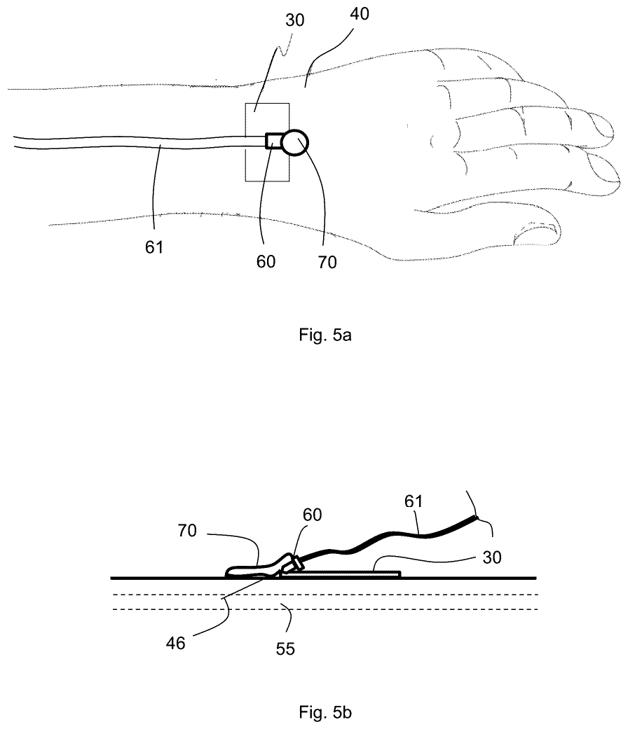

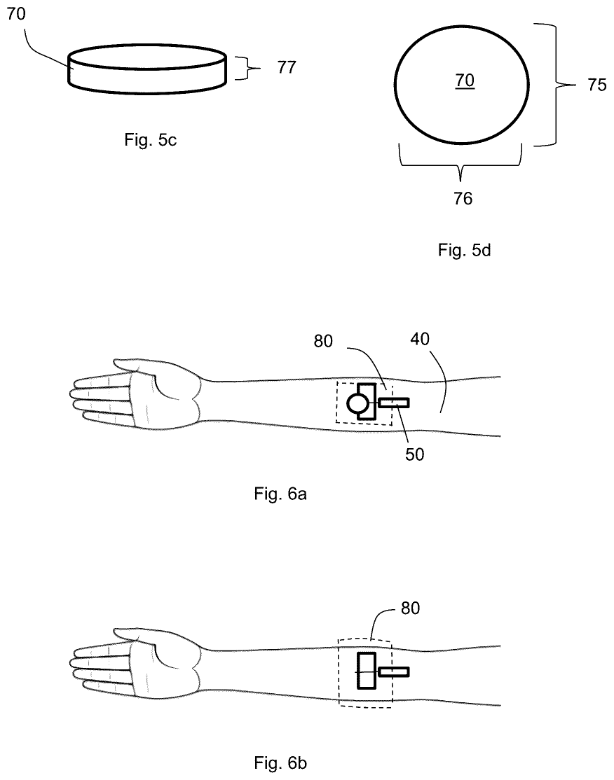

Optionally, the needle tip can be covered by antimicrobial patch 70, as shown in FIGS. 5a and 5b. The term “antimicrobial” refers to the ability of a material to inhibit the growth of or kill microorganisms, such as bacteria, yeast, fungi, mycoplasma, viruses, virus-infected cells, protozoa, etc. Thus, the puncture site is protected, lowering the chance of infection.

Patch 70 can be constructed from any suitable material, such as (but not limited to) rubber, silicone, plastic, fiber, fabric, cotton, foam, gel, and the like. In some embodiments, the material used to construct the patch is absorbent to absorb any blood or serum that oozes from the skin puncture site. The patch can further include one or more antibacterial agents. Suitable antimicrobial materials can include (but are not limited to) chlorhexidine base, chlorhexidine acetate, chlorhexidine gluconate, EDTA, silver sulfadiazine, taurolidine, essential oils, copper, copper nanoparticles, citric acid, acetic acid, or combinations thereof.

The patch can have any suitable size and/or shape. For example, the patch can have a square, rectangular, oval, circular, triangular, hexagonal, pentagonal, etc. shape. Further, the patch can have length 75 and/or width 76 of about 0.25-1 inch (e.g., at least/no more than about 0.25, 0.3, 0.35, 0.4, 0.45, 0.5, 0.55, 0.6, 0.65, 0.7, 0.75, 0.8, 0.85, 0.9, 0.95, or 1 inch. Similarly, thickness 77 of patch 70 can be about 0.01-0.25 inches (e.g., at least/no more than about 0.01, 0.1, 0.15, 0.2, 0.25 inches), as shown in FIGS. 5c and 5d. It should be appreciated that the dimensions of the patch are not limited and can be configured outside the ranges given herein.

The disclosed method further includes securing the injection site with non-adhesive covering 80. Specifically, a portion of non-adhesive covering can be used to cover the anchor, catheter, and optional antimicrobial patch. The term “non-adhesive covering” can include any covering that does not adhesively attach to the skin of the patient (or to the anchor, catheter, or patch 70). Thus, covering 80 can be constructed from saran, PVC, polyurethane, nylon polyurethane, fabric, woven or non-woven material, nylon, polyethylene terephthalate, and the like.

In some embodiments, non-adhesive covering 80 can be transparent or semi-transparent. The term “transparent” refers to a material that has the property of transmitting visible light without appreciable scattering, such that an object placed beyond the transparent material is visible. The term “semi-transparent” refers to a material that has the property of transmitting visible light with some appreciable scattering, such that an object placed beyond the transparent material is at least partially visible. In these embodiments, the patient and/or health care provider can see through the covering and monitor the proper placement of the catheter. In some embodiments, the materials used to construct the covering can be flexible, allowing it to bend and give as the patient moves without breaking or being damaged.

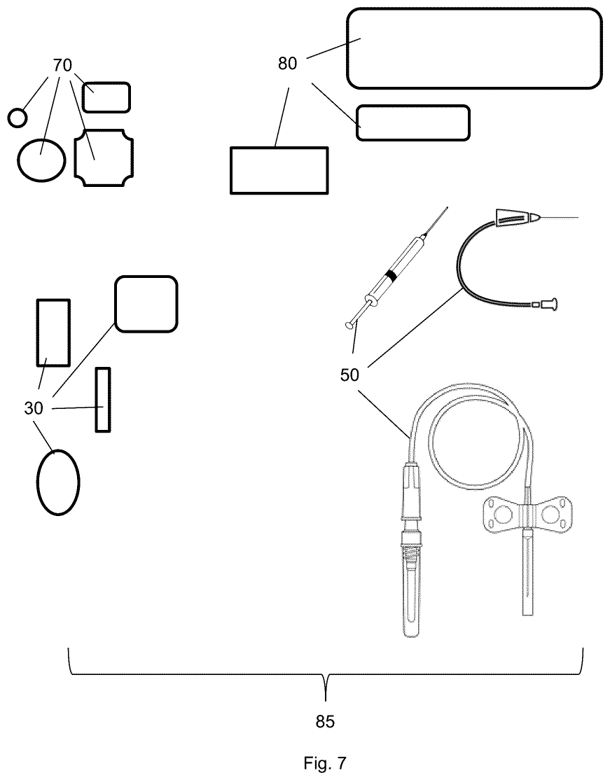

The covering can be partially or fully wrapped around a patient's limb, as shown in FIGS. 6a and 6b. Any suitable method can be used to secure covering 80 in place, such as the use of clips, magnets, fasteners, static electricity, buttons, and the like. Thus, the covering can releasably attach to itself without the use of adhesive.

In use, covering 80 preserves the sterile state of the injection site to reduce the likelihood of infection. The full infusion line and the head of the infusion needle or catheter can be checked or inspected. Any shock to the infusion line, such as by patient pulling can be at least partially absorbed by covering 80 in an area that is isolated from the needle or catheter. In this way, pulling on the infusion line is less likely to cause (or can prevent) the infusion needle or catheter to be disturbed. In addition, the head of the infusion needle or catheter is secured by covering so that it cannot be disturbed or moved. Further the injection site is continuously protected by covering 80 from any exposure to bacteria or other contaminants.

When the health care provider determines that the catheter is no longer needed, removal is quick and efficient. Specifically, non-adhesive covering 80 is removed from the patient's arm. Because covering 80 is non-adhesive, removal is painless and can be done by simply unwrapping the covering, lifting the cover, unhooking straps, unhooking fasters, or combinations thereof. When present, the anti-microbial patch can then be removed from the application site by applying a lifting motion. The needle can be removed from vessel 55, as well as the non-adhesive tape 30. Because non-adhesive tape and non-adhesive covering 65 are used, the patient's skin is irritation-free, especially compared to conventional methods that promote the use of adhesive materials that stay in contact with the user's skin for prolonged periods of time.

In some embodiments, the materials used to perform the disclosed method can be available in kit form. As shown in FIG. 6, kit 85 can include a plurality of anchors 30 in different shapes and sizes. The kit can optionally include antimicrobial patches 70, each varying in type of antibiotic used, material used, size, shape, and the like. The kit also includes catheter 50 that vary in shape, size, and/or purpose. The kit further includes non-adhesive coverings 80 that can vary by shape, size, material used, and the like. The user can then select from different materials for each patient. In some embodiments, instructions for using the disclosed method are included or printed on the kit packaging. The kit can be packaged in a sterile condition. The term “sterile” refers to the condition of being substantially free of foreign matter or undesired microorganisms. In some embodiments, the kit and/or materials used in the disclosed method are of medical grade sterility, which is typically set by a regulatory agency.

The term “patient” as used herein refers to human patients (e.g., adults, children, the elderly). The disclosed system and method can also be applied for veterinary use, such that the term “patient” can include pets and livestock. Thus, the term “patient” can include (but is not limited to) humans, dogs, cats, rabbits, hamsters, gerbils, guinea pigs, horses, llamas, alpacas, cows, goats, pigs, donkeys, birds, reptiles, and the like.

The disclosed system and method include many advantages over conventional methods of inserting a catheter and/or IV line in patient. For example, because no adhesive materials are used in contact with the patient's skin, little to no skin irritation occurs when the method is applied. Stated another way, the anchor and covering do not pull, tear, or irritate the patient's skin when being applied, when in place, or when removed.

In addition, when the anchor and non-adhesive covering are removed, there is no pain associated with pulling skin or hair from the patient.

The disclosed system is secure, ensuring that the catheter remains in proper position, even when the patient moves or while sleeping. Particularly, the anchor and outer covering ensure that the needle remains in place.

The antimicrobial patch offers additional advantages, reducing the likelihood of infection at the injection site. The patch can further provide stability to the injection site, keeping the needle in proper position.

The disclosed method is efficient and can be performed quickly and easily. The difficulty and time-consuming task of having to remove pieces of adhesive tape carefully and when the infusion needle needs to be inspected has been eliminated.

The disclosed method is easy to use, and medical staff can be quickly and easily trained to use the method on a wide variety of patients.

Exemplary embodiments of the methods and components of the presently disclosed subject matter have been described herein. As noted elsewhere, these embodiments have been described for illustrative purposes only, and are not limiting. Other embodiments are possible and are covered by the presently disclosed subject matter. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

What is claimed is:

1. A method of securely inserting a catheter in a vessel of a patient, the method comprising:
applying an anchor on a patient's skin, adjacent to a vessel, wherein the anchor is defined as a portion of non-adhesive material;
inserting a catheter needle into the vessel at an injection site such that the needle partially overlays the anchor;
wrapping an outer covering around the anchor and catheter needle at the injection site, wherein the outer covering is defined as a portion of non-adhesive material;
wherein the catheter is securely inserted in the patient's vessel.

2. The method of claim 1, further comprising applying an antimicrobial patch over the injection site after the needle has been inserted and prior to wrapping the outer covering.

3. The method of claim 1, wherein the anchor and outer covering each have a peel strength of less than 0.01 N/20 mm.

4. The method of claim 1, wherein the anchor and outer covering do not adhere to the skin of the patient.

5. The method of claim 1, wherein the anchor comprises a bacteriostatic or bactericidal agent.

6. The method of claim 1, wherein the anchor is constructed from hydrocolloid, acrylic PSA, silicone, elastomer, rubber, polyurethane, or combinations thereof.

7. The method of claim 1, wherein the anchor has a length and width of about 0.5-4 inches and a thickness of at least about 0.001 inches.

8. The method of claim 1, wherein the catheter includes a needle, a hub, and tubing.

9. The method of claim 2, wherein the antimicrobial patch includes chlorhexidine base, chlorhexidine acetate, chlorhexidine gluconate, EDTA, silver sulfadiazine, taurolidine, or combinations thereof.

10. The method of claim 1, wherein the outer covering is transparent.

11. The method of claim 1, wherein the outer covering is semi-transparent.

12. The method of claim 1, wherein the outer covering comprises saran, PVC, polyurethane, nylon polyurethane, nylon, polyethylene terephthalate, or combinations thereof.

13. The method of claim 1, wherein the outer covering is flexible.

14. The method of claim 1, wherein the outer covering wraps fully around a patient's limb.

15. The method of claim 1, wherein the outer cover releasably attaches to itself.

16. A kit for performing the method of claim 1, the kit comprising:
a plurality of anchors that vary in size, shape, composition, or combinations thereof;
a plurality of catheters that vary in size, shape, purpose, composition, or combinations thereof;
a plurality of outer coverings that vary in size, shape, composition, or combinations thereof.

17. The kit of claim 16, further comprising a plurality of antimicrobial patches that vary in size, shape, composition, or combinations thereof.

18. The kit of claim 16, wherein the outer covering is transparent or semi-transparent.

19. The kit of claim 16, wherein the outer covering comprises saran, PVC, polyurethane, nylon polyurethane, nylon, polyethylene terephthalate, or combinations thereof.

20. The kit of claim 16, wherein the anchor comprises a bacteriostatic or bactericidal agent.
